(19) United States
(12) Patent Application Publication
(10) Pub. No.: US 2024/0301336 A1
(43) Pub. Date: Sep. 12, 2024

(54) PROTECTIVE CARRIER FOR MICROCAVITY VESSELS

(71) Applicant: CORNING INCORPORATED, CORNING, NY (US)

(72) Inventors: Thomas Albert Cloutier, Arundel, ME (US); William Joseph Lacey, North Andover, MA (US); Ana Maria del Pilar Pardo, Portsmouth, NH (US); Allison Jean Tanner, Portsmouth, NH (US)

(21) Appl. No.: 18/571,999

(22) PCT Filed: Jun. 14, 2022
(86) PCT No.: PCT/US2022/033351

§ 371 Date: Dec. 19, 2023

Related U.S. Application Data
(60) Provisional application No. 63/216,754, filed on Jun. 30, 2021.

(51) Int. Cl. C12M 1/24; C12M 1/00; C12M 1/32

(52) U.S. Cl. CPC C12M 23/08; C12M 23/12; C12M 23/38

(57) ABSTRACT

A protective carrier for a microcavity vessel is provided. The protective carrier comprises a substantially flat rigid plate comprising a top surface and a bottom surface. A flange disposed around at least a portion of a perimeter of the rigid plate, the flange extending from the top surface of the rigid plate. One or more ribs disposed on the bottom surface of the rigid plate. A protective microcavity vessel carrier system is provided that comprises a microcavity vessel comprising a microcavity substrate on a bottom surface; and a protective carrier. The microcavity vessel may be a microcavity flask, microcavity plate such as an open well reservoir plate, microcavity bioreactor, or a stacked 3D microcavity culture vessel.

### CROSS-REFERENCE TO RELATED APPLICATIONS

This application claims the benefit of priority under 35 U.S.C. § 119 of U.S. Provisional Application Ser. No. 63/216,754 filed on Jun. 30, 2021, the content of which is relied upon and incorporated herein by reference in its entirety.

### TECHNICAL FIELD

This disclosure generally relates to cell culture devices and methods. In particular, the present disclosure relates to three-dimensional (3D) cell culture and protective carriers for microcavity vessels.

### BACKGROUND

Cells cultured in 3D environments exhibit more in vivo-like functionality than cells cultured in two-dimensional (2D) environments as monolayers. In 2D cell culture systems, cells attach to a substrate on which they are cultured. In contrast, when grown in 3D systems, cells interact with each other rather than attaching to the substrate to form 3D cell cultures or cell aggregates, such as spheroids or organoids. However, culturing or generating a large amount of uniform cell aggregates presents difficulties. While cell non-adherent surfaces may promote cell aggregate formation in large-scale culture vessels, such as low-adhesion or no-adhesion surfaces in CellSTACK® culture chambers (Corning Incorporated, Corning, NY), cell aggregates formed in such large-scale culture vessels are not homogeneous.

Cell culture device formats commercially available for the formation of homogeneous cell aggregates are limited to microcavity vessels that have micron-sized wells. To allow generation of large numbers of cell aggregates, microcavity vessels may have a large common culture area for the production of large numbers of homogeneous cell aggregates and may include features directed to gas permeability, seeding density, growth time, risk of displacing or dislodging spheroid clusters, or a combination thereof. Such features, however, may make the microcavity vessels susceptible to damage during shipping and use.

### SUMMARY

In aspect of the subject matter described herein, a device is provided that protects the gas permeable microcavities of microcavity vessels during shipping and that can also act as a protective carrier for microcavity vessels during use for cell culture. The protective carrier is comprised of a rigid plate that is slightly larger than a footprint of a bottom of a microcavity vessel. In embodiments, the microcavity vessel comprises a microcavity substrate on a bottom surface of the microcavity vessel. In some embodiments, the microcavity vessel may be a microcavity flask. In some embodiments, the microcavity vessel may be a microcavity plate, such as an open well microcavity plate. In some embodiments, the microcavity vessel may further comprise a lid.

In an aspect, the disclosure is directed to a protective carrier for a microcavity vessel. In some embodiments, the protective carrier comprises a substantially flat rigid plate comprising a top surface and a bottom surface; a flange disposed around at least a portion of a perimeter of the rigid plate, the flange extending from the top surface of the rigid plate; and one or more ribs disposed on the bottom surface of the rigid plate.

In some embodiments, the flange is disposed continuously around the perimeter of the top surface of the rigid plate.

In some embodiments, the flange is disposed around the perimeter of the top surface of the rigid plate on all but one side of the rigid plate.

In some embodiments, a footprint of the protective carrier is substantially rectangular. In some embodiments, the footprint of the protective carrier comprises rounded corners.

In some embodiments, the one or more ribs disposed on the bottom surface of the rigid plate is proximate the perimeter of the rigid plate. In some embodiments, the one or more ribs has a height of about 1 mm-2 mm extending from the bottom surface of the rigid plate.

In an aspect, embodiments of the protective carrier further comprise a plurality of standoffs. In some embodiments, each standoff of the plurality of the standoffs is disposed at an interior surface of the flange on the top surface of the rigid plate.

In some embodiments, each standoff is an “L” shape, wherein a first portion is disposed vertically on an interior surface of the flange and a second portion is disposed horizontally on the top surface of the rigid plate. In some embodiments, the first portion of each standoff has a height of about 3 mm-4 mm and a thickness of about 0.5 mm-1.5 mm. In some embodiments, the second portion of each standoff has a length of about 3 mm-4 mm and a thickness of about 0.5 mm-1.5 mm.

In some embodiments, the protective carrier is configured to retain a bottom portion of a microcavity vessel, the bottom portion of the microcavity vessel disposed at the top surface of the rigid plate within the perimeter defined by the flange. In some embodiments, the plurality of standoffs is configured to support the bottom portion of the microcavity vessel, allowing for air exchange to a microcavity substrate of the microcavity vessel. In some embodiments, the microcavity vessel is a microcavity flask, microcavity plate, microcavity bioreactor, or a stacked 3D microcavity culture vessel. In some embodiments, the microcavity vessel comprises a microcavity flask. In some embodiments, the microcavity vessel comprises a microcavity plate.

In some embodiments, the protective carrier has a height of about 7 mm to 8 mm. In some embodiments, a thickness of the rigid plate is about 1 mm-2 mm.

In some embodiments, the protective carrier is formed from a polymer, metal, or glass. In some embodiments, the polymer comprises polystyrene, polypropylene, polyethylene, polyethylene terephthalate, polymethylpentene, polycarbonate, polymethyl methacrylate, styrene-ethylene-butadiene-styrene, other such polymers, or a combination thereof. In some embodiments, the metal comprises aluminum, stainless steel, zinc, or a combination thereof. In some embodiments, the glass comprises a borosilicate glass.

In some embodiments, the protective carrier is formed from a recyclable material. In some embodiments, the protective carrier is formed from a biodegradable material. In some embodiments, the protective carrier is opaque. In some embodiments, the protective carrier is translucent.

In an aspect, an embodiment of the present disclosure is directed to a protective microcavity vessel carrier system comprising a microcavity vessel comprising a microcavity substrate on a bottom surface; and a protective carrier according to embodiments described herein.

In some embodiments, the microcavity vessel is a microcavity flask, microcavity plate, microcavity bioreactor, or a stacked 3D microcavity culture vessel.

In some embodiments, the microcavity substrate comprises a plurality of microcavities. In some embodiments, the plurality of microcavities are arranged in a hexagonal close-pack pattern. In some embodiments, each microcavity comprises a rounded bottom. In some embodiments, each microcavity is configured such that cells cultured in the microcavity vessel form three-dimensional (3D) cell aggregates.

In some embodiments, an interior surface of the microcavity substrate is non-adherent to cells. In some embodiments, the interior surface of the microcavity substrate comprises a cell non-adherent surface coating comprising perfluorinated polymers, olefins, lipids, agarose, non-ionic hydrogels, polyethers, polyols, polymers that inhibit cell attachment, or a combination thereof. In some embodiments, the cell non-adherent surface coating comprises an ultra-low attachment (ULA) surface coating.

In some embodiments, the microcavity substrate is formed from polydimethylsiloxane (PDMS), polymethylpentene, (poly)4-methylpentene (PMP), polyethylene(PE), polystyrene (PS), polypropylene, polyethylene terephthalate, polycarbonate, polymethyl methacrylate, styrene-ethylene-butadiene-styrene, a silicone rubber or copolymer, ethylene vinyl acetate, polysulfone, polytetrafluoroethylene, poly(styrene-butadiene-styrene), or a combination thereof.

### BRIEF DESCRIPTION OF THE DRAWINGS

FIG. 1 shows an embodiment of a protective platen for a microplate.

FIG. 2 shows an image of an embodiment of a microcavity substrate of a microcavity vessel.

FIG. 3 shows a close-up view of an embodiment of a microcavity substrate.

FIG. 4 shows a close-up view of an embodiment of a microcavity substrate.

FIG. 5 shows a close-up view of an embodiment of a microcavity substrate.

FIG. 6 shows a perspective view of a protective carrier according to an embodiment.

FIG. 7 shows a perspective view of a protective carrier with microcavity vessel according to an embodiment.

FIG. 8 shows a perspective view of a protective carrier with microcavity vessel according to an embodiment.

FIG. 9 shows a cross-sectional side view of a protective carrier with microcavity vessel according to an embodiment.

FIG. 10 shows a perspective view of a protective carrier according to an embodiment.

FIG. 11 shows a perspective view of a protective carrier with microcavity vessel according to an embodiment.

FIG. 12 shows a perspective view of a protective carrier with microcavity vessel according to an embodiment.

FIG. 13 shows a cross-sectional side view of a protective carrier with microcavity vessel according to an embodiment.

FIG. 14 shows a bottom view of an embodiment of a protective carrier.

FIG. 15 shows a perspective view of a protective carrier with microcavity vessel according to an embodiment.

FIG. 16 shows a perspective view of an embodiment of a protective carrier.

FIG. 17 shows top view of an embodiment of a protective carrier.

FIG. 18 shows a cross-sectional side view at line C-C of the protective carrier embodiment of FIG. 17.

FIG. 19 shows a cross-sectional side view at line D-D of the protective carrier embodiment of FIG. 17.

### DETAILED DESCRIPTION

Reference will now be made in greater detail to various embodiments of the subject matter of the present disclosure, some embodiments of which are illustrated in the accompanying drawings. Like numbers used in the figures refer to like components, steps, and the like. However, it will be understood that the use of a number to refer to a component in a given figure is not intended to limit the component in another figure labeled with the same number. In addition, the use of different numbers to refer to components is not intended to indicate that the different numbered components cannot be the same or similar to other numbered components.

Gas permeability is a property that contributes to the 3D cell culture environment. By allowing for gas permeability within microcavities of the microcavity vessel, cell culture growth media may need to be changed out less frequently and cell growth may be encouraged. Microcavity vessels are unique in their geometry and formation in that they are formed from a gas permeable substrate with micron-scale wells, also referred to as a microcavity substrate. Such microcavity vessels enjoy gas permeability due to the thickness of the microcavity substrate, wherein gas permeability occurs because the microcavity substrate is formed from a very thin polystyrene material of manufacture, which has a thickness of about 28 micrometers to about 72 micrometers. Though gas permeability may be an asset for culturing cell aggregates, the thinness of the microcavity substrate material makes microcavity cell culture vessels susceptible to damage during shipping and use.

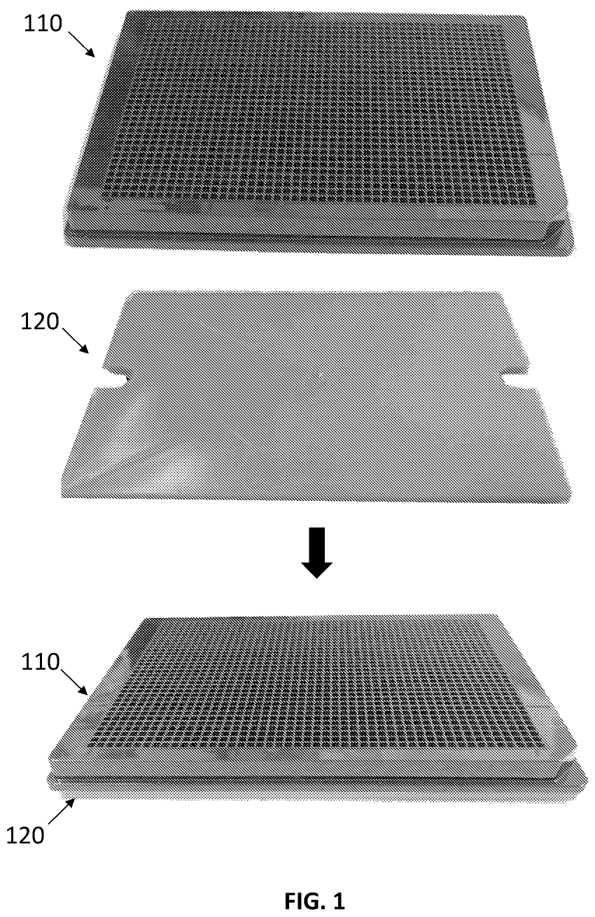

As shown in FIG. 1, some microplates 110, such as a 1536 well microplate (Corning Incorporated, Corning, NY), may include a protective platen 120 during shipping. The platen 120 is sized to fit into a bottom recess of microplate 110 to prevent scuffing of the bottom of the wells of the microplate during shipping. However, such a platen would not protect a vessel with microcavities. For example, because the platen 120 is configured to fit inside the footprint of the microplate, there is no room or space for gas exchange, and such a platen is not intended to be retained on the microplate during use or for carrying the microplate.

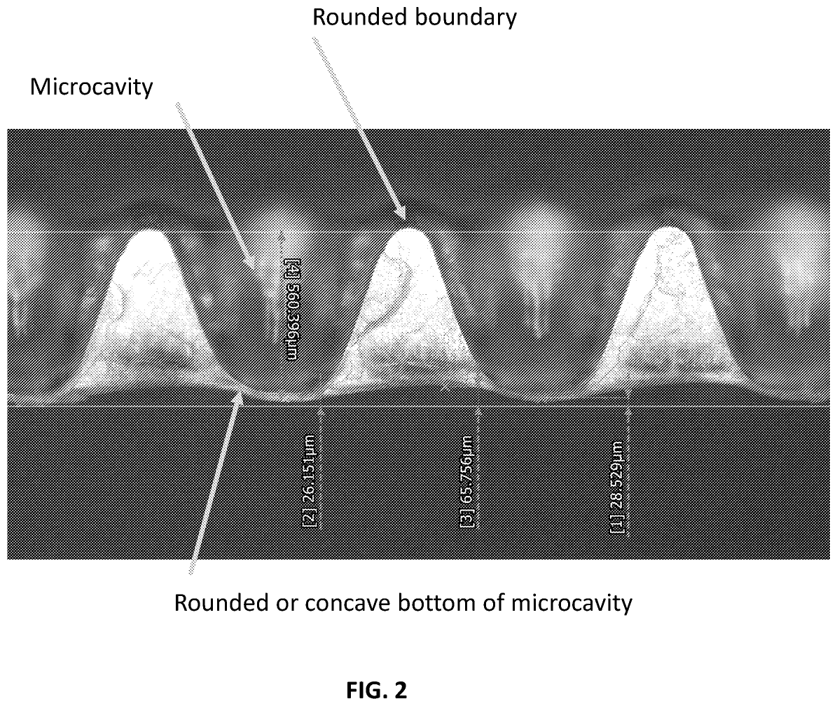

FIG. 2 shows an image of a cross-section of a microcavity substrate of a microcavity vessel according to an embodiment. As shown in FIG. 2, the microcavity substrate may have a cross-sectional shape that is undulating or is a shape approximating a sine wave. In such embodiments, the bottom of the microcavity well is rounded (e.g., hemispherically round), the side walls increase in diameter from the bottom of the well to the top and the boundary or barrier between wells is rounded. As such, the top of the microcavity wells does not terminate at a right angle. In some embodiments, the width of the well is greater than the width of the barrier between contiguous wells. Such an embodiment permits a greater number of wells within a given area of culture surface.

The microcavity substrate embodiment shown in FIG. 2 shows microcavity proportions and the thickness of the substrate along the bottoms of each microcavity. The diameter of the microcavities is approximately 500 microns. The depth of a microcavity well in the microcavity substrate of FIG. 2 was measured to be 560.396 micrometers, the measurement taken from a center of the microcavity well opening at a top of the boundary between wells, to a center of the microcavity well bottom. The thickness of the microcavity substrate of FIG. 2 measured at the bottom of the microcavity wells is in a range from 26.151 micrometers to 65.756 micrometers, depending on where the thickness is measured within the microcavity bottom. When the thickness of the microcavity substrate of FIG. 2 was measured at a bottom of a microcavity well where the well bottom meets the sidewall of the well, the thickness measurement was 65.756 micrometers. When the thickness of the microcavity substrate of FIG. 2 was measured at a center of the bottom of the microcavity well, the thickness range was 26.15 micrometers to 28.529 micrometers.

Microcavity vessels according to embodiments described herein comprise microcavity substrates comprising a plurality of microcavities. Each microcavity may include an inner cavity with a rounded bottom that is non-adherent to cells. Thus, microcavity vessels as described herein are cell culture devices facilitate 3D cell culture by allowing cells seeded into the microcavities to self-assemble or attach to one another to form a spheroid in each microcavity. Microcavities may be shallow and permit cell culture medium to cover the spheroids, organoids, or 3D cell aggregates in all cavities at once to make manual handling easy.

In an embodiment, a top plane of the microcavities may be recessed to a location close to a bottom of the sidewalls. Individual microcavities may hold a small volume of medium. The individual microcavities may have any suitable dimensions. For example, the diameter or width of individual microcavities may be in a range of about 500 microns to about 5 millimeters. The depth of individual microcavities may be in a range of about 500 microns to about 6 millimeters. In some embodiments, a depth of the individual microcavities may be about 500 microns to about 650 microns. In some embodiments, a depth of the individual microcavities may be about 1.6 millimeters. An excess of culture medium may be added to the microcavity vessel so that the spheroids, organoids, or 3D cell aggregates do not need to rely only on the small amount of medium in the individual microcavities.

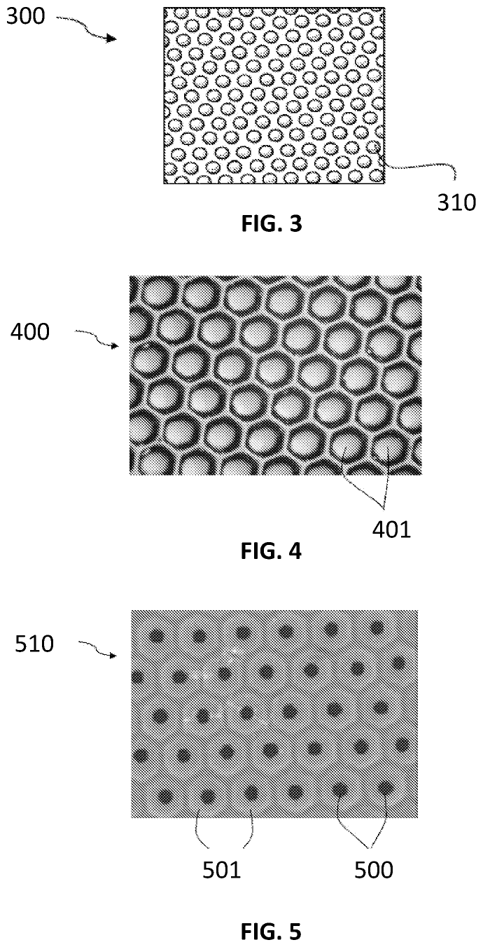

FIG. 3 shows a close-up view of an embodiment of a microcavity substrate patterned with an array of microwells, forming a bottom surface of a microcavity vessel. The close-up view of the microcavity substrate 300 comprises an array of microcavities 310 or microwells. Such a microcavity substrate having an array of microcavities or microwells as described herein may define any suitable number of microcavities that may have any suitable size or shape. The microcavities define a volume based on their size and shape. In many embodiments, one or more or all of the microcavities are symmetric and/or symmetrically rotatable around a longitudinal axis. In some embodiments, the longitudinal axes of one or more or all of the microcavities are parallel with one another. The microcavities may be uniformly or non-uniformly spaced. In some embodiments, the microcavities are uniformly spaced. One or more or all the microcavities can have the same size and shape or can have different sizes and shapes.

In some embodiments, the microcavity substrate defining the microcavities includes an array of hexagonal close-packed microcavities. Such hexagonal close-packing density or “honeycomb” microcavity configuration, combined with the micron-sized geometry of the microcavities, allows for many spheroids, organoids, or 3D cell aggregates to be cultured at once, resulting in bulk spheroid, organoid, or 3D cell aggregate production. An image of an embodiment of such a substrate 400 is shown in FIG. 4, showing the substrate having an array of hexagonal microcavities 401. In an embodiment, such packing density allows for approximately 12,588 wells that are 500 μm in diameter in a typical microplate working surface area of about 4.5 inches by about 3 inches. FIG. 5 shows cells (spheroids, organoids, or 3D cell aggregates) 500 grown in microcavities 501 of an embodiment of a substrate 510 having an array of microcavities having a hexagonal close-packed microcavity structure. In some embodiments, the cells within each microcavity 501 form a single spheroid, organoid, or 3D cell aggregate 500, as depicted.

Microcavity vessels according to embodiments of the disclosure provide a homogenous culturing environment. All spheroids, organoids, or 3D cell aggregates cultured in the microcavity vessels may receive the same treatment at the same time, thereby providing a homogenous culture environment. In contrast, typical plates with individual wells have more of a heterogenous culture environment because dispensing the same volume to each well is difficult, even with automated equipment.

In aspect of the subject matter described herein, a device is provided that protects the gas permeable microcavities of microcavity vessels during shipping and that can also act as a protective carrier for microcavity vessels during use for cell culture. The protective carrier is comprised of a rigid plate that is slightly larger than a footprint of a bottom of a microcavity vessel. In embodiments, the microcavity vessel comprises a microcavity substrate on a bottom surface of the microcavity vessel. In some embodiments, the microcavity vessel may be a microcavity flask. In some embodiments, the microcavity vessel may be a microcavity plate, such as an open well microcavity plate or reservoir microcavity plate. In some embodiments, the microcavity vessel may be stackable and may further comprise a lid or top surface.

In an embodiment, a protective carrier is provided for protecting a delicate microcavity substrate on a bottom of a microcavity vessel. In an aspect, the protective carrier may be formed of a rigid material that will support a microcavity vessel. The protective carrier may comprise a substantially flat rigid plate with a flange extending upwards from a top surface of the rigid plate and one or more ribs extending downwards from a bottom surface of the rigid plate. If the protective carrier is warped or twisted, a bottom portion of a microcavity vessel may not fit within or on the protective carrier. The substantially flat rigid plate may comprise a substantially rectangular shape. In some embodiments, the rigid plate may comprise a substantially rectangular shape with rounded corners. The rigid plate may have any suitable thickness that allows a user to handle and transport the protective carrier while supporting a microcavity vessel. In some embodiments, the rigid plate has a thickness of about 1 mm to about 2 mm. In some embodiments, the rigid plate has a thickness of about 1.5 mm to 1.8 mm.

In an embodiment, the protective carrier may comprise a raised flange around a perimeter of a top surface of the rigid plate that conforms to the shape at the bottom perimeter of the microcavity vessels. The flange on the rigid plate may have a gap or opening in it so that microcavity vessels can be manipulated for filling and emptying while still within the protective carrier. In some embodiments, the rigid plate area may be extended to act as a handle offering a place to grip the carrier with the vessel in place, or there may be no gap in the flange. In some embodiments, the rigid plate area comprises an extension on one side of the protective carrier to form a handle. The flange may have any suitable height that allows for a microcavity vessel to be received and supported by the protective carrier. In some embodiments, the flange has a height of about 5 mm to about 6 mm. In some embodiments, the protective carrier has a total height of about 7 mm to about 8 mm.

In an embodiment, the protective carrier may comprise a plurality of raised stand-offs inside the flange on the top surface of the rigid plate. The stand-offs elevate the microcavity vessel off of the top surface of the protective carrier to permit gas exchange to occur between the microcavities and ambient air. The raised stand-offs keep the bottom of the microcavity vessel from having intimate contact with the top surface of the rigid plate. The space created between the microcavity vessel and the top surface of the rigid plate due to the stand-offs allows for gas exchange through the gas permeable substrate of the microcavity vessel. For example, the standoffs allow for oxygen to get between the carrier and the microcavity substrate, allowing for gas exchange. In some embodiments, each standoff has a thickness of about 0.5 mm to about 1.5 mm. In some embodiments, each standoff comprises a first portion that extends from the top surface of the rigid plate at a height of about 3 mm to 4 mm. In some embodiments, the height of the first portion is about 3 mm to about 3.5 mm. In some embodiments, each standoff comprises a second portion that extends from the interior side of the flange along the top surface of the rigid plate at a length of about 3 mm to about 4 mm. In some embodiments, the length of the second portion of the standoff is about 3 mm to about 3.5 mm. In some embodiments, the protective carrier comprises standoffs disposed proximate to corners of the substantially rectangular protective carrier.

In an embodiment, the protective carrier may comprise one or more ribs on a bottom surface of the rigid plate of the protective carrier. The one or more ribs extending downward from the bottom surface of the rigid plate raise the protective carrier off of the surface on which it sits. The one or more ribs act as feet on the bottom of the protective carrier. By providing the protective carrier with ribs extending from the bottom surface of the rigid plate, a space is provided between the protective carrier and the surface on which it sits. This feature allows for circulation of air underneath the protective carrier and makes the protective carrier easier to pick up. The ribs or feet allow for easier transport and ease of use, as the feet help to break up a vacuum that may exist between the bottom surface of the protective carrier and a work surface, such as a lab bench or incubator surface. For example, if the bottom surface of the carrier is flat, a vacuum may form between the flat bottom surface of the protective carrier and a flat work surface. Such a vacuum may present difficulty for a user, including issues when attempting to move or pick up the protective carrier with microcavity vessel from the work surface, which may require precarious dragging of the carrier with vessel to an edge of a surface to break the vacuum.

The ribs may be any suitable height to allow airflow circulation. In some embodiments, the one or more ribs has a height of about 1 mm to about 2 mm. In some embodiments, the one or more ribs extends around a perimeter of the bottom surface about 1 mm to about 2 mm from the edge of the bottom surface. In some embodiments, the one or more ribs is a continuous rib. In some embodiments, the one or more ribs has a larger offset from the edge of the bottom surface at the sides of the rigid plate compared to the offset of the one or more ribs proximate the corners of the rigid plate. In some embodiments, the offset of the one or more ribs is configured to allow stacking of a first protective carrier on top of a second protective carrier without the one or more ribs of the first protective carrier being in intimate contact with standoffs of the second protective carrier.

In an embodiment, the protective carrier may comprise any suitable shape that follows a footprint of a microcavity vessel to be received by the protective carrier. In some embodiments, the protective carrier may comprise a substantially rectangular shape. In some embodiments, the protective carrier has a width of about 85 mm to about 90 mm. In some embodiments, the protective carrier has a width of about 87 mm to about 88 mm. In some embodiments, the protective carrier has a length of about 120 mm to about 125 mm. In some embodiments, the protective carrier has a length of about 122 mm to about 123 mm. In some embodiments, the substantially rectangular shape of the protective carrier may have rounded corners. In some embodiments, the shape of the protective carrier may comprise a substantially rectangular portion to receive a footprint of a microcavity vessel and an area of the rigid plate that extends past the footprint in order to act as a handle for a user.

In an aspect, a top of the protective carrier is configured to mate with a bottom of a 3D culture vessel, such as a microcavity flask or a microcavity plate. A microcavity flask may have feet on a bottom portion of the microcavity flask, and the feet at the bottom portion may rest on the plurality of standoffs to allow gas exchange and space between the flange and top surface of the protective carrier.

In an aspect, a bottom of the protective carrier is configured to mate with a top of a 3D culture vessel, such as a microcavity flask or a microcavity plate. In some embodiments, the bottom of the protective carrier is configured to mate with a lid at a top surface of a microcavity flask, allowing for stacking of microcavity flasks and protective carriers. Such stacking may be beneficial for use of space in an incubator or on a lab bench. The microcavity flask may also have a ridge or lip on a lid at a top of the microcavity flask, and the one or more ribs on the bottom surface of the protective carrier may be configured to mate with the ridge or lip on the lid of the microcavity flask to allow for stacking.

The protective carrier may be made from any material suitable to provide a rigid support for a microcavity vessel. In some embodiments, the protective carrier may be formed from polymer material, metal, glass, or a combination thereof. Polymer materials of construction for the protective carrier may comprise a “plastic” polymer, co-polymer, or polymer blend. Nonlimiting examples of polymer materials include polystyrene, polypropylene, polyethylene, polyethylene terephthalate, polymethylpentene, polycarbonate, polymethyl methacrylate, styrene-ethylene-butadiene-styrene, other such polymers, or a combination thereof. In some embodiments, the protective carrier is formed from polypropylene. Nonlimiting examples of metal materials include aluminum, stainless steel, zinc, or a combination thereof. Nonlimiting examples of glass materials include borosilicate glass or Gorilla® glass (Corning Incorporated, Corning, NY). In some embodiments, the protective carrier may be formed from a material so that the carrier is translucent or clear. In some embodiments, the protective carrier may be formed from a material so that the carrier is colored or opaque. In some embodiments, the protective carrier may be formed from a material that is recyclable or reusable. In some embodiments, the protective carrier may be formed from a material that is biodegradable. Nonlimiting examples of recyclable or biodegradable materials include polymer materials such as polystyrene, polypropylene, polyethylene, polyethylene terephthalate, polymethylpentene, polycarbonate, polymethyl methacrylate, styrene-ethylene-butadiene-styrene, other such polymers, or a combination thereof.

In an aspect, the protective carrier according to embodiments described herein may be used to transport or carry the microcavity vessel. The thin microcavity substrate is protected by the rigid protective carrier, and the rigid protective carrier provides more surface area for a user to hold and/or carry the microcavity vessel without risk of damaging the thin microcavity substrate.

In an aspect, the protective carrier according to embodiments described herein may be used during shipping to protect the thin microcavity substrate at a bottom of the microcavity vessel. The protective carrier is configured to allow a bottom portion of the microcavity vessel to mate with a top portion of the protective carrier. The protective carrier may comprise a rigid plate having a top surface with a flange disposed around at a least a portion of the perimeter of the plate, the flange extending upwards from the top surface of the rigid plate. Standoffs may be disposed around the interior surface of the flange and on the top surface of the rigid plate. In embodiments, the microcavity vessel comprises a bottom surface having a thin microcavity substrate with feet extending downward from the bottom surface, such as on at least a portion of the perimeter of the microcavity vessel. The feet disposed on the bottom portion of the microcavity vessel fit within flange of the protective carrier and rest or sit on the standoffs of the protective carrier. The standoffs allow for a space or separation between the sides and the bottom of the microcavity vessel, and the flange and top surface of the protective carrier.

In an aspect, the protective carrier according to embodiments described herein is suitable for use in a cell culture incubator or environment with constant temperature and humidity sufficient for growth of tissue culture cells. The microcavity vessel mates with the protective carrier to protect the thin microcavity substrate at the bottom of the microcavity vessel against scuffing from an incubator surface. The protective carrier also allows for oxygen to access the microcavity substrate at the bottom of the microcavity vessel, allowing for gas exchange for the microcavity vessel.

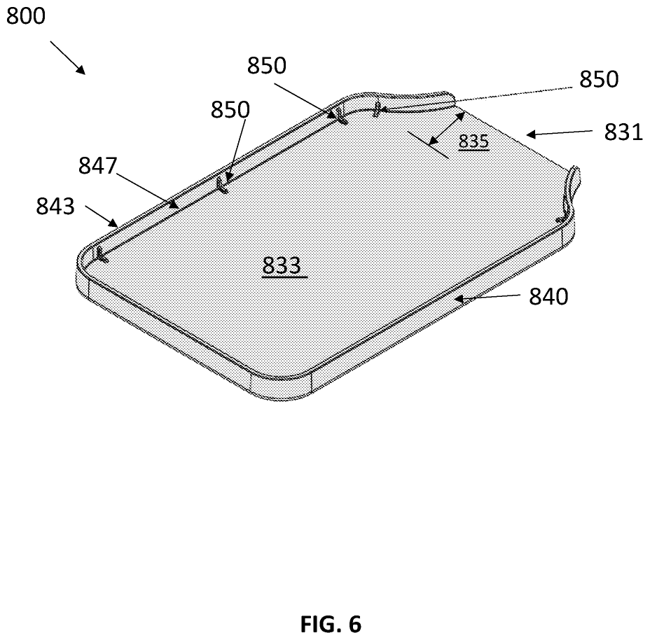

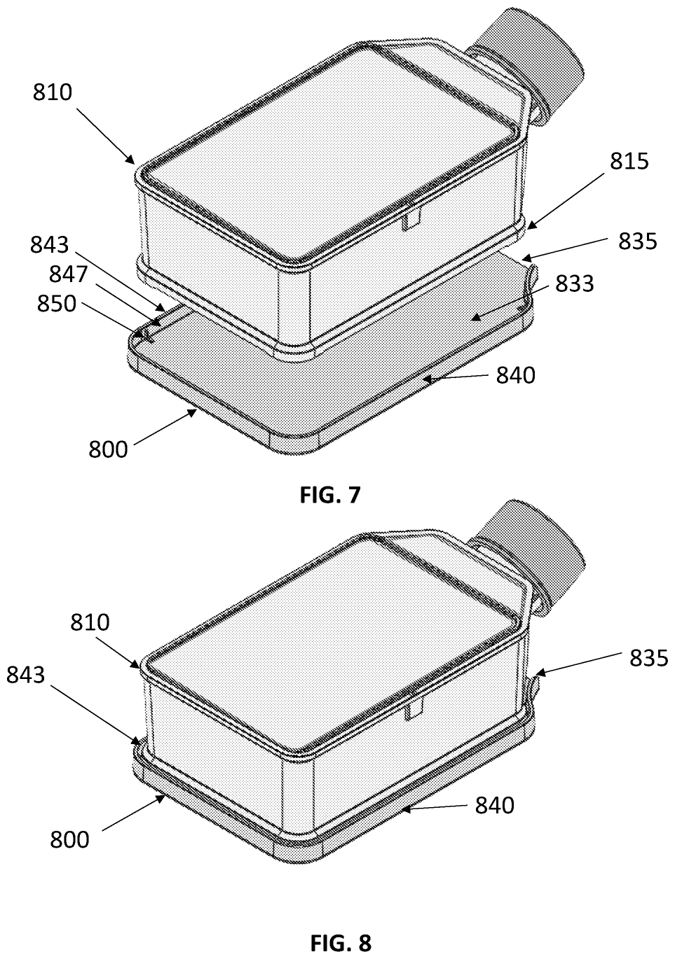

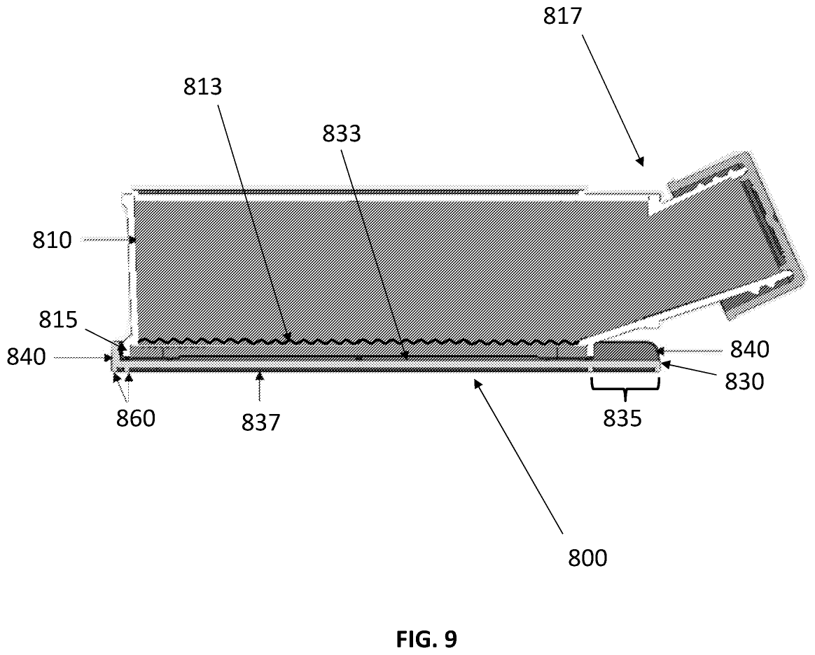

FIG. 6 to FIG. 9 show an embodiment of a protective carrier 800 for a microcavity vessel. FIG. 6 shows a perspective view of a protective carrier 800 according to an embodiment FIG. 7 shows a perspective view of protective carrier 800 with a microcavity vessel 810 suspended over the protective carrier 800. FIG. 8 shows a perspective view of the protective carrier 800 and the microcavity vessel 810 of FIG. 7 wherein the microcavity vessel 810 is in place on the protective carrier 800. FIG. 9 shows a cross-sectional side view of the microcavity vessel 810 is in place on the protective carrier 800.

The protective carrier 800 comprises a rigid plate 830 having a top surface 833 and a bottom surface 837. A flange 840 extends upwards from the top surface 833 around at least a portion of a perimeter of the protective carrier 800. In the embodiment of the protective carrier shown in FIGS. 6-9, the flange 840 does not extend continuously around the perimeter of the protective carrier 800 and there is a gap in the flange 840 at one side 831 of the protective carrier 830 The handle 835 is an area of the protective carrier that extends past the portion of the protective carrier that receives the bottom portion 815 or footprint of the microcavity vessel 810 towards the side 831 of the rigid plate 830 without a flange 840. The handle 835 may be any suitable shape or size. In an embodiment, the handle 835 may have a width less than the width of the opposite side of the protective carrier 800. As shown in FIGS. 7-9, a front portion 817 of a microcavity vessel 810, such as a microcavity flask, may be positioned at the gap in the flange 840 at a side 831 of the protective carrier 800. In some embodiments, the front portion 817 of a microcavity vessel 810 may extend past a handle 835 of the protective carrier 800.

A plurality of standoffs 850 are disposed on a top surface 833 of the protective carrier 800, each standoff 850 comprising a first portion extending vertically from the top surface 833 in intimate contact with an interior surface 847 of the flange 840, and a second portion extending from the first portion in a horizontal direction along, and in intimate contact with, the top surface 833. In some embodiments, the first portion of each standoff 850 does not extend vertically past the top 843 of flange 840. A bottom portion 815 of the microcavity vessel 810 may rest against the first portion of the standoffs 850 and on top of the second portion of the standoffs 850. Thus, the microcavity vessel may be disposed on top of the protective carrier and within the flange forming at least a part of the perimeter of the protective carrier. Because the bottom portion 815 of the microcavity vessel 810 rests on, or is supported by, the standoffs 850, the bottom of the microcavity vessel comprising the microcavity substrate 813 is not in intimate contact with the top surface 833 of the rigid plate 830. The standoffs 850 allow for space between the top surface 833 of the protective carrier 800 and the microcavity substrate 813, as well as space between the interior surface 847 of the flange 840 and the bottom portion 815 of the microcavity vessel 810. The space created by the standoffs 850 allows for the fragile microcavity substrate 813 to be protected by the substantially flat rigid plate. The space created by the standoffs 850 also allows for air exchange through the gas permeable microcavity substrate 813 of the microcavity vessel 810.

The protective carrier 800 comprises one or more ribs 860 on the bottom surface 837 of the rigid plate 830. The one or more ribs 860 may be a continuous rib. The one or more ribs 860 may extend as a continuous rib proximate a perimeter of the bottom surface 837, about 1 mm to about 2 mm from the edge of the bottom surface. The one or more ribs 860 extends downward from the bottom surface 837 of the rigid plate 830 and acts as feet for the protective carrier 800. By providing the protective carrier 800 with one or more ribs 860 extending from the bottom surface 837, a space is provided between the protective carrier 800 and a surface, such as a horizontal surface (e.g. a lab bench, an incubator surface) upon which the protective carrier may sit, thereby allowing for circulation of air underneath the protective carrier.

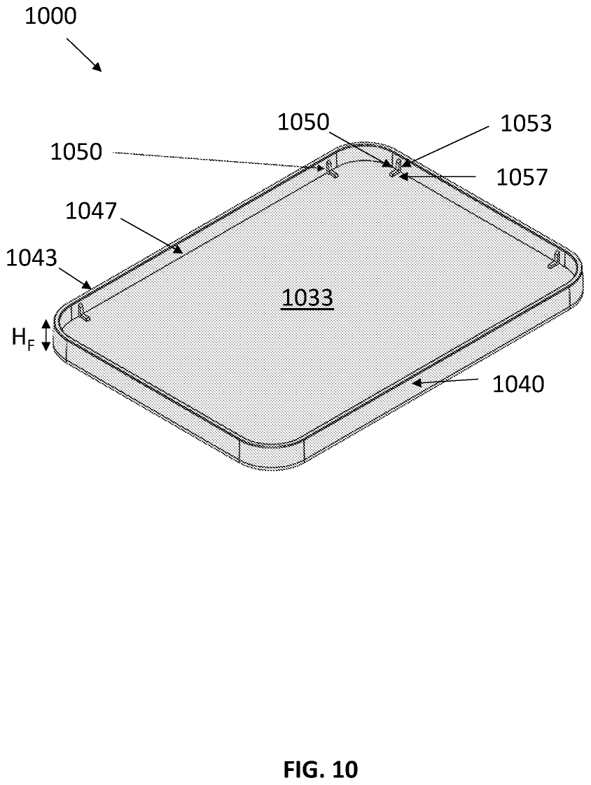

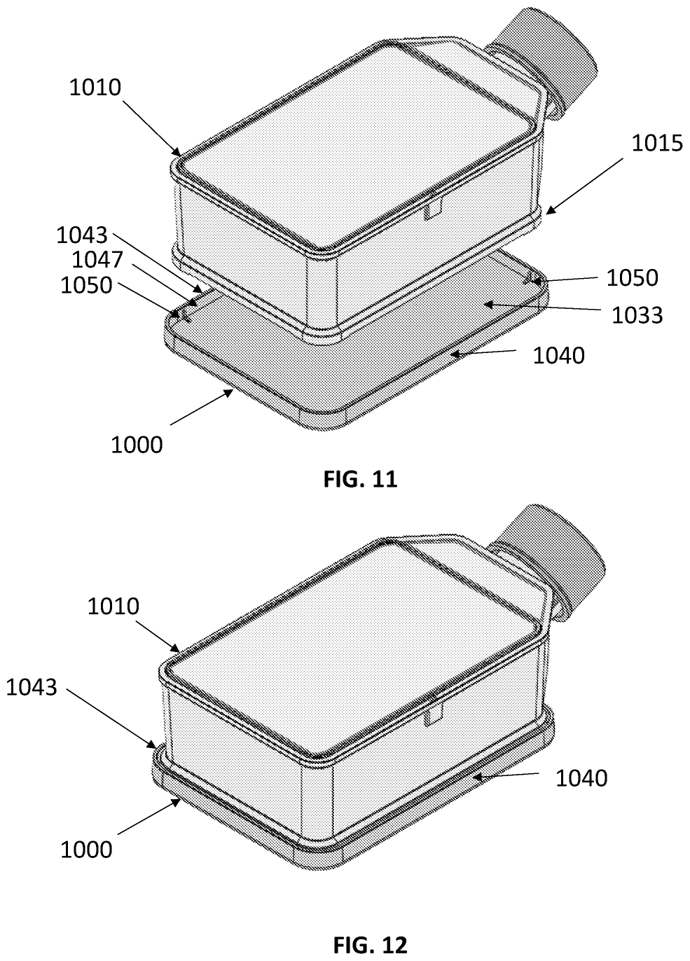

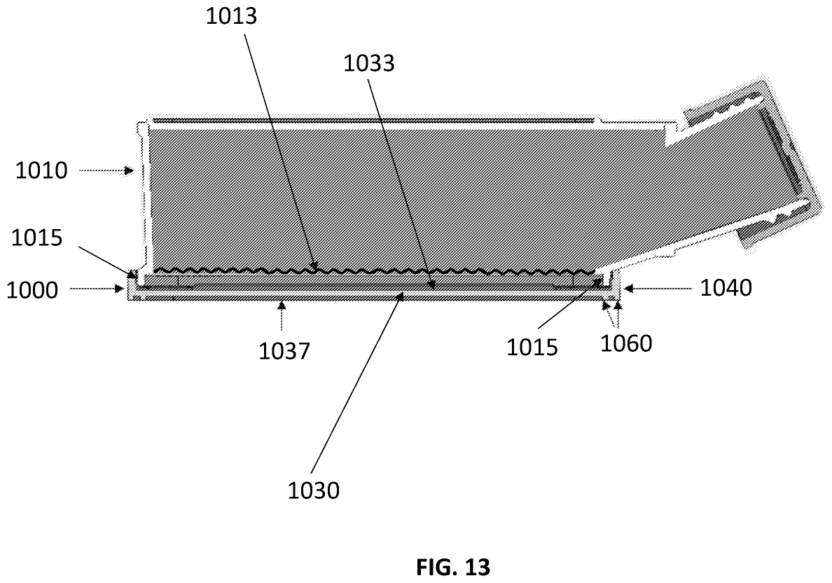

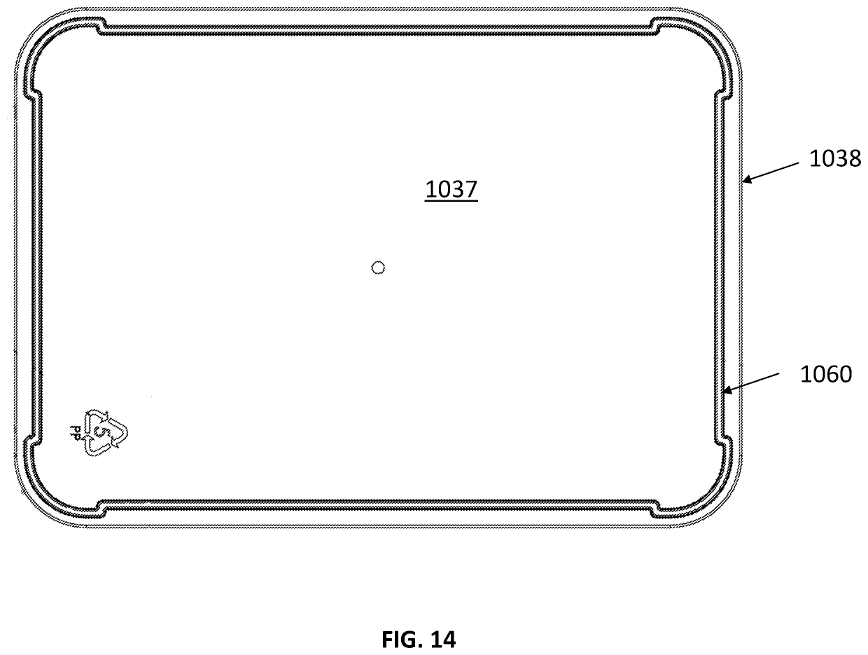

FIG. 10 to FIG. 14 show an embodiment of a protective carrier 1000 for a microcavity vessel. FIG. 10 shows a perspective view of a protective carrier 1000 according to an embodiment. FIG. 11 shows a perspective view of protective carrier 1000 with a microcavity vessel 1010 suspended over the protective carrier 1000. FIG. 12 shows a perspective view of the protective carrier 1000 and the microcavity vessel 1010 of FIG. 11 wherein the microcavity vessel 1010 is in place on the protective carrier 800. FIG. 13 shows a cross-sectional side view of the microcavity vessel 1010 is in place on the protective carrier 1000. FIG. 14 shows a bottom view of the protective carrier 1000.

The protective carrier 1000 comprises a rigid plate 1030 having a top surface 1033 and a bottom surface 1037. A flange 1040 extends upwards from the top surface 1033 around a perimeter 1038 of the protective carrier 1000. In the embodiment of the protective carrier shown in FIGS. 10-14, the flange 1040 extends continuously around the perimeter of the protective carrier 1000. Due to the rigid material that forms the protective carrier, a user is able to pick the protective carrier up from the sides or hold on to the bottom surface of the protective carrier while transporting or during use without damaging the delicate microcavity substrate on a bottom of a microcavity vessel disposed in the protective carrier.

A plurality of standoffs 1050 are disposed on a top surface 1033 of the protective carrier 1000, each standoff 1050 comprising a first portion extending vertically from the top surface 1033 in intimate contact with an interior surface 1047 of the flange 1040, and a second portion extending from the first portion in a horizontal direction along, and in intimate contact with, the top surface 1033. In some embodiments, the first portion of each standoff 850 does not extend vertically past the top 1043 of flange 1040. A bottom portion 1015 of the microcavity vessel 810 may rest against the first portion of the standoffs 1050 and on top of the second portion of the standoffs 1050. Thus, the microcavity vessel may be disposed on top of the protective carrier and within the flange forming the perimeter of the protective carrier. Because the bottom portion 1015 of the microcavity vessel 1010 rests on, or is supported by, the standoffs 1050, the bottom of the microcavity vessel comprising the microcavity substrate 1013 is not in intimate contact with the top surface 1033 of the rigid plate 1030. The standoffs 1050 allow for space between the top surface 1033 of the protective carrier 1000 and the microcavity substrate 1013, as well as space between the interior surface 1047 of the flange 1040 and the bottom portion 1015 of the microcavity vessel 1010. The space created by the standoffs 1050 allows for the fragile microcavity substrate 1013 to be protected by the substantially flat rigid plate. The space created by the standoffs 1050 also allows for air exchange through the gas permeable microcavity substrate 1013 of the microcavity vessel 1010.

The protective carrier 1000 comprises one or more ribs 1060 on the bottom surface 1037 of the rigid plate 1030. As shown in FIG. 14, the one or more ribs 1060 is a continuous rib 1060. The one or more ribs 1060 extends as a continuous rib proximate the perimeter 1038 of the bottom surface 1037, about 1 mm to about 2 mm from the edge of the bottom surface. The one or more ribs 1060 has a larger offset from the perimeter 1038 or edge of the bottom surface 1037 at the sides of the rigid plate compared to the offset of the one or more ribs proximate the corners of the rigid plate. The one or more ribs 1060 extends downward from the bottom surface 1037 of the rigid plate 1030 and acts as feet on the bottom of the protective carrier 1000. By providing the protective carrier 1000 with one or more ribs 1060 extending from the bottom surface 1037, a space is provided between the protective carrier 1000 and a surface, such as a horizontal surface (e.g. a lab bench, an incubator surface) upon which the protective carrier may sit. This feature allows for circulation of air underneath the protective carrier and makes the protective carrier easier to pick up.

In an aspect of the subject matter described herein, the protective carrier may have dimensions suitable for receiving a microcavity vessel comprising a microcavity plate. In some embodiments, the microcavity plate may comprise an open well plate. For example, a microcavity open well plate may include a bottom plate or bottom surface and one or more sidewalls. In an embodiment, the microcavity open well plate may comprise a bottom plate defining a major surface, one or more sidewalls extending from the bottom plate defining a reservoir, and a plurality of microcavities formed in the major surface. The bottom plate may be formed, in whole or in part, from a substrate having an array of microcavities that promote or induce the growth of spheroids, organoids, or 3D cell aggregates. Each microcavity defines an upper aperture co-planar with the major surface and open to the reservoir, and a microcavity-bottom nadir positioned below the major surface. The open well microcavity plate may define a reservoir above the surface of the microcavities, which allows for increased volumes of cell culture media to be used and thus provides for less frequent media exchange. Reservoir plates may permit the addition of culture medium in excess of what would be typically used to fill individual shallow wells of a microwell plate and may allow cells cultured in different microcavities to be in fluid communication.

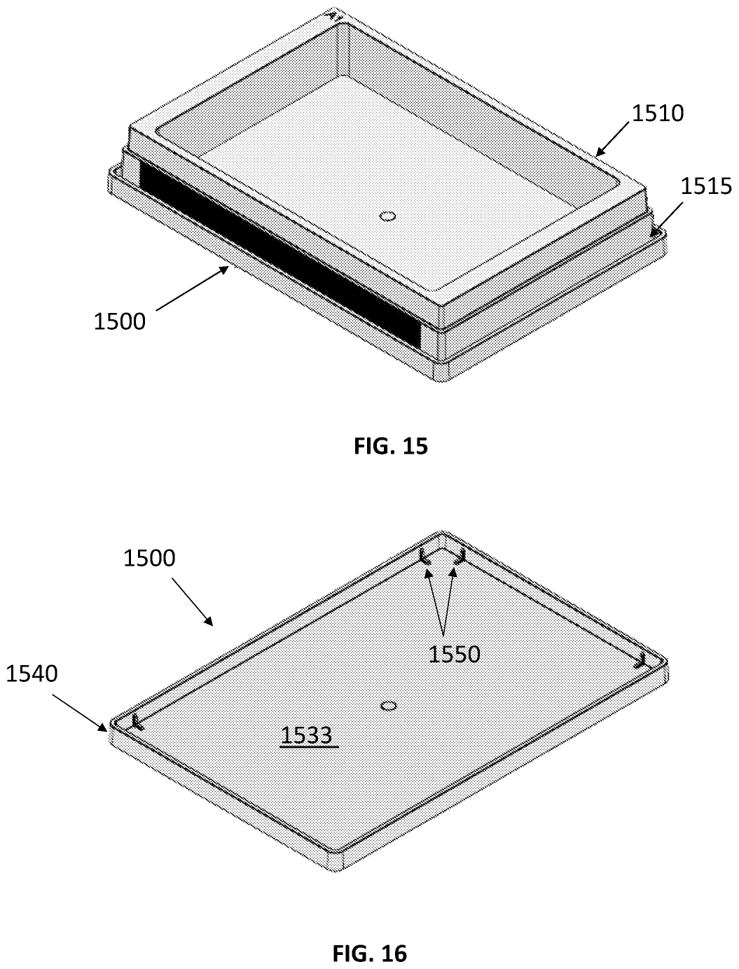

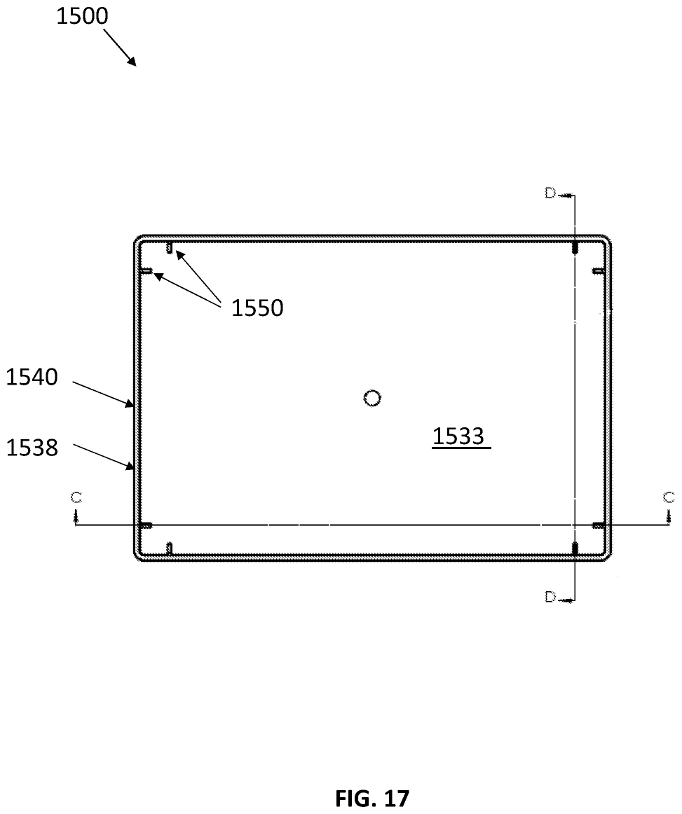

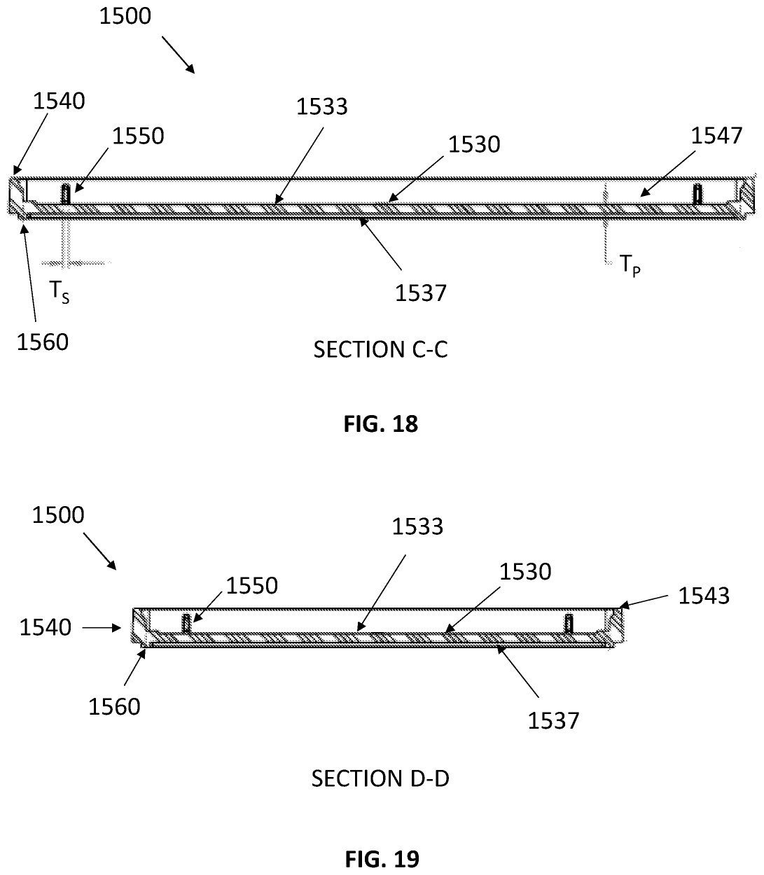

FIG. 15 to FIG. 19 show an embodiment of a protective carrier 1500 with a microcavity vessel 1510 such as a microcavity open well plate. FIG. 15 shows a perspective view of protective carrier 1500 with microcavity vessel 1510 according to an embodiment. FIG. 16 shows a perspective view of protective carrier 1500. FIG. 17 shows a top view of protective carrier 1500 FIG. 18 shows a cross-sectional side view at line C-C of the protective carrier 1500. FIG. 19 shows a cross-sectional side view at line D-D of the protective carrier 1500. As seen in FIGS. 15-19, the protective carrier is configured to receive the microcavity vessel 1510, the protective carrier 1500 having a footprint slightly larger than a bottom portion 1515 of the microcavity vessel 1510. The protective carrier 1500 comprises a substantially rectangular rigid plate 1530 having a perimeter 1538, a top surface 1533 and a bottom surface 1537. A flange 1540 is disposed at a perimeter 1538 of the rigid plate 1530 and extends upwards from the top surface 1533. A plurality of standoffs 1550 are disposed around the flange 1540, a first portion of each standoff 1550 extending vertically from the top surface 1533 and in intimate contact with the interior surface 1547 of the flange 1540, and a second portion of each standoff 1550 extending horizontally from the first portion along the top surface 1533 of the rigid plate 1530. In embodiments, the protective carrier may have any suitable number of standoffs that will support a microcavity vessel and allow for air exchange to the microcavity substrate of the microcavity vessel. The standoffs may be arranged around the flange on the top surface at any suitable locations. In some embodiments, the standoffs may be arranged uniformly around the protective carrier. In some embodiments, the standoffs may be arranged proximate to each corner of the rigid plate, alongsides of the rigid plate, or a combination thereof. In embodiments, the thickness of each standoff T_{S} will be any suitable thickness that will support a microcavity vessel and allow for air exchange to the microcavity substrate of the microcavity vessel. In some embodiments, each standoff of the plurality of standoffs of the protective carrier will have a same T_{S}.

In an aspect, the protective carriers as described herein may be used to angle a cell culture vessel during liquid handling steps. Such angling of the cell culture vessel allows for spheroid retention during the liquid handling steps. For example, the protective carrier may be positioned on a flat surface, and a portion of the cell culture vessel may be positioned on the protective carrier. Furthermore, a first portion of the cell culture vessel (e.g., a first side of the cell culture vessel, a first corner of the cell culture vessel) may be positioned on the protective carrier and a second portion of the cell culture vessel (e.g., a second side of the cell culture vessel opposite the first side; a second corner of the cell culture vessel opposite the first corner of the cell culture vessel) is positioned on the flat surface, thereby providing the cell culture vessel which angles downwards from the first portion positioned on the protective carrier to the second portion positioned on the flat surface. Liquid handling steps may include media exchange (e.g., media removal from, and addition to, the cell culture vessel).

In an embodiment, the cell culture vessel may be positioned at least partly on the protective carrier during liquid handling steps of cell culture. In an embodiment, the liquid handling step comprises cell culture media exchange. In an embodiment, cell culture media exchange comprises addition of cell culture media to the cell culture vessel, removal of cell culture media from the cell culture vessel, or a combination thereof. The protective carrier may be positioned or placed on a flat surface. The cell culture vessel may then be placed or positioned at least partly on the flat surface and partly on the protective carrier. In an embodiment, a first corner of the cell culture vessel may be positioned on the protective carrier and a second corner of the cell culture vessel that is opposite from the first corner may be placed or positioned on the flat surface. In an embodiment, a first side of the cell culture vessel may be positioned on the protective carrier and a second side of the cell culture vessel that is opposite from the first side may be placed or positioned on the flat surface. Thus, the arranged position provides an angled cell culture vessel, wherein the cell culture vessel is angled downward from the first portion of the cell culture vessel (i.e., the first corner or the first side) arranged or positioned on the protective carrier down to the second portion of the cell culture vessel (i.e., the second corner or the second side) arranged or positioned on the flat surface.

Microcavity vessels according to embodiments of the disclosure may be any suitable vessel comprising microcavities for use in cell culture. Nonlimiting examples of microcavity vessels may include a microcavity flask, microcavity plate such as an open well reservoir plate, microcavity bioreactor, or a stacked 3D microcavity culture vessel.

Microcavity vessels according to embodiments of the disclosure provide a homogenous culturing environment. All spheroids, organoids, or 3D cell aggregates cultured in the microcavity plate may receive the same treatment at the same time, thereby providing a homogenous culture environment. In contrast, typical plates with individual wells have more of a heterogenous culture environment because dispensing the same volume to each well is difficult, even with automated equipment.

In certain embodiments, microcavity vessels described herein comprise a microcavity substrate as a bottom surface. The microcavity substrate comprises a plurality of microcavities. In some embodiments, the microcavities are in gaseous communication with an exterior of the microcavity vessel via gas permeable materials of the microcavity substrate. Each microcavity in the plurality of microcavities may be configured to cause cells cultured in the microcavities to form spheroids, organoids, or 3D cell aggregates of a specified diameter. The microcavities may be any size suitable for culturing spheroids, organoids, or 3D cell aggregates. In some embodiments, the width of the microcavities may be in a range from about 500 microns wide to about 5 mm in width. In some embodiments, the depth of the microcavities may be in a range from about 500 microns deep to about 6 mm deep. For example, in embodiments with the larger size microcavities, the microcavities overlap with spheroid plate well sizes, thereby allowing for organoid development in bulk culture.

In some embodiments, the microcavity shape transitions to alleviate issues with air-escape upon introduction of liquid into the microcavities. In some embodiments, a circular cross-section microcavity bottom (or bottom portion of the microcavity) may be optimal for spheroid formation but problematic for air escape without pocket formation. To alleviate this issue, microcavities may be formed with a circular well-bottom cross-section and a non-circular (e.g., triangular, square, rectangular, pentagonal, hexagonal, etc.) top aperture. In such embodiments, the sidewalls transition from the non-circular (e.g., polygonal) top aperture to the circular microcavity bottom. In some embodiments, the transition is a gradual one, so as to not introduce any interfering, jagged, or horizontal-presenting microcavity sidewall features that could result in the ‘hanging up’ of air bubbles escaping the microcavity upon introduction of liquid to the microcavity. In some embodiments, the corners in the microcavity sidewalls created by the non-circular (e.g., polygonal) shape of the transitioning walls and top aperture provide pathways for the entry of liquid and/or the escape of air.

The microcavity substrate may be formed from the same material or a similar material and method for making the rest of the microcavity vessel. In some embodiments, the microcavity substrate may be molded or formed separately from the rest of the microcavity vessel and bonded subsequently through thermal-bonding, ultrasonic welding, or any other method of plastic joining. The material of construction for the microcavity vessel, microcavity substrate, or both may comprise a “plastic” polymer, co-polymer, or polymer blend. Nonlimiting examples include silicone rubber, polystyrene, polypropylene, polyethylene, polyethylene terephthalate, polymethylpentene, polycarbonate, polymethyl methacrylate, styrene-ethylene-butadiene-styrene, other such polymers, or a combination thereof. Any suitable construction method may be used to form the microcavity substrate, such as nonlimiting examples include injection molding thermoforming, 3D printing, or any other method suitable for forming a plastic part.

In some embodiments, gas-permeable/liquid impermeable materials are used in construction of microcavity substrates, microcavity vessels, or both. Nonlimiting examples of gas-permeable/liquid impermeable materials include polystyrene, polycarbonate, ethylene vinyl acetate, polysulfone, polymethylpentene (PMP), polytetrafluoroethylene (PTFE) or compatible fluoropolymer, a silicone rubber or copolymer, poly(styrene-butadiene-styrene), or polyolefin, such as polyethylene or polypropylene, or combinations of these materials. Microcavity substrates may be formed of any suitable material having a suitable gas permeability over at least a portion of the well. Nonlimiting examples of suitable microcavity substrates include polydimethylsiloxane (PDMS), polymethylpentene, (poly)4-methylpentene (PMP), polyethylene(PE), polystyrene (PS), polypropylene, polyethylene terephthalate, polycarbonate, polymethyl methacrylate, styrene-ethylene-butadiene-styrene, a silicone rubber or copolymer, ethylene vinyl acetate, polysulfone, polytetrafluoroethylene, poly(styrene-butadiene-styrene), or a combination thereof. Such materials allow effective gas exchange between the microcavity cell culture area and the outside atmosphere to allow the ingress of the oxygen and other gases, while preventing the passage of liquid or contaminants.

In some embodiments, the thickness of microcavity substrate material is adjusted to allow for optimized gas exchange. The thickness of the microcavity substrate may be dependent on the material of construction. In some embodiments, microcavity bottom thickness is between 10 and 75 μm (e.g., 10 μm, 15 μm, 20 μm, 25 μm, 30 μm, 35 μm, 40 μm, 45 μm, 50 μm, 60 μm, 70 μm, 75 μm, and any ranges there between). In embodiments, the microcavities may have an oxygen transmission rate through the microcavity substrate gas permeable polymeric material of 2000 cc/m²/day or greater. In some embodiments, the microcavities may have a gas permeability through the substrate of 3000 cc/m²/day or greater. In some embodiments, the microcavities may have a gas permeability through the substrate of 5000 cc/m²/day or greater.

In some embodiments, the microcavities are structured and arranged to provide an environment that is conducive to the formation of spheroids, organoids, or 3D cell aggregates in culture. That is, in embodiments, the microcavities have spheroid-inducing geometry. For example, the microcavities in which cells are grown can be non-adherent to cells to cause the cells in the microcavities to associate with each other and form spheroids, organoids, or 3D cell aggregates. The spheroids expand to size limits imposed by the geometry of the microcavities. Uniform geometry of the microcavities allows cells grown therein to form similar-sized cell aggregates or spheroids. In some embodiments, the cell culture substrate in the devices is non-adherent to cells to cause the cells to associate with each other instead of the substrate. The combination of non-adherent microcavities, spheroid-inducing microcavity geometry, and gravity can define a confinement volume in which growth of cells cultured in the microcavities is limited, which results in the formation of spheroids having dimensions defined by the confinement volume.

In some embodiments, the inner surface of the microcavities, or cell culture surface, comprise a low-adhesion or no-adhesion material and/or are coated with a low-adhesion or no-adhesion material. For example, in some embodiments, inner surfaces of the microcavities or cell culture surface may be coated or treated with polymers or lipids that inhibit cell attachment in order to prevent cell attachment. Nonlimiting examples of such polymer or lipid treatments include poly-HEMA treatment, pluronic treatment, treatment with a lipid low adhesion treatment such as Lipidure®-CM5206 powder (Amsbio, Cambridge, MA), or treatment with an ultra-low binding material such as an ultra-low attachment (ULA) material. Examples of no-adhesion or non-adherent materials include perfluorinated polymers, olefins, lipids, or like polymers or mixtures thereof. Other examples include agarose, non-ionic hydrogels such as polyacrylamides, polyethers such as polyethylene oxide and polyols such as polyvinyl alcohol, or like materials or mixtures thereof. In some embodiments, the low-binding treatment or surface coating is a Corning® Ultra Low Attachment (ULA) surface coating (Corning Incorporated, Corning, NY). The Corning® ULA (Corning Incorporated, Corning, NY) surface is hydrophilic, biologically inert and non-degradable, which promotes highly reproducible spheroid formation and easy harvesting. The covalent attachment of the ULA surface reduces cellular adhesion to the well surface. The ULA surface allows for uniform and reproducible 3D multicellular spheroid formation.

A wide variety of cell types may be cultured in microcavity vessels described herein. For example, any type of cell may be cultured on embodiments of microcavity vessels described herein including, but not limited to, immortalized cells, primary culture cells, cancer cells, stem cells (e.g., embryonic or induced pluripotent), etc. The cells may be mammalian cells, avian cells, piscine cells, etc. The cells may be in any cultured form including disperse (e.g., freshly seeded), confluent, 2-dimensional, 3-dimensional, spheroid, etc. The cultured cells may further be used in a wide variety of research, diagnostic, drug screening and testing, therapeutic, and industrial applications.

In some embodiments, the cells are mammalian cells (e.g., human, mice, rat, rabbit, dog, cat, cow, pig, chicken, goat, horse, etc.). The cells may be of any tissue type including, but not limited to, kidney, fibroblast, breast, skin, brain, ovary, lung, bone, nerve, muscle, cardiac, colorectal, pancreas, immune (e.g., B cell), blood, etc. Cells may be from or derived from any desired tissue or organ type, including but not limited to, adrenal, bladder, blood vessel, bone, bone marrow, brain, cartilage, cervical, corneal, endometrial, esophageal, gastrointestinal, immune system (e.g., T lymphocytes, B lymphocytes, leukocytes, macrophages, and dendritic cells), liver, lung, lymphatic, muscle (e.g., cardiac muscle), neural, ovarian, pancreatic (e.g., islet cells), pituitary, prostate, renal, salivary, skin, tendon, testicular, and thyroid. In some embodiments, the cell is a somatic cell. In some embodiments, the cell is a stem cell or progenitor cell (e.g., embryonic stem cell, induced pluripotent stem cell) in any desired state of differentiation (e.g., pluripotent, multi-potent, fate determined, immortalized, etc.) In some embodiments, the cell is a disease cell or disease model cell. For example, in some embodiments, the spheroid comprises one or more types of cancer cells or cells that can be induced into a hyper-proliferative state (e.g. transformed cells).

In some embodiments, the systems, devices, and methods herein comprise one or more cells. In some embodiments, the cells are cryopreserved. In some embodiments, the cells are in three-dimensional culture. In some such embodiments, the systems, devices, and methods comprise one or more spheroids. In some embodiments, one or more of the cells are actively dividing. In some embodiments, a spheroid contains a single cell type. In some embodiments, a spheroid contains more than one cell type. In some embodiments, where more than one spheroid is grown, each spheroid is of the same type, while in other embodiments, two or more different types of spheroids are grown. Cells grown in spheroids may be natural cells or altered cells (e.g., cell comprising one or more non-natural genetic alterations).

Any cell culture medium capable of supporting the growth of cells may be used when culturing cells using cell culture devices described in embodiments herein. Cell culture medium may be for example, but is not limited to, sugars, salts, amino acids, serum (e.g., fetal bovine serum), antibiotics, growth factors, differentiation factors, colorant, or other desired factors. Exemplary cell culture medium includes Dulbecco's Modified Eagle Medium (DMEM), Ham's F12 Nutrient Mixture, Minimum Essential Media (MEM), RPMI Medium, Iscove's Modified Dulbecco's Medium (IMDM), MesenCult™-XF medium (commercially available from STEMCELL Technologies Inc.), and the like.

In some embodiments, the systems, devices, and methods comprise culture media (e.g. comprising nutrients (e.g., proteins, peptides, amino acids), energy (e.g., carbohydrates), essential metals and minerals (e.g., calcium, magnesium, iron, phosphates, sulphates), buffering agents (e.g., phosphates, acetates), indicators for pH change (e.g., phenol red, bromo-cresol purple), selective agents (e.g., chemicals, antimicrobial agents), etc.). In some embodiments, one or more test compounds (e.g., drug) are included in the systems, devices, and methods.

It will be appreciated that the various disclosed embodiments may involve particular features, elements, or steps that are described in connection with that particular embodiment. It will also be appreciated that a particular feature, element, or step, although described in relation to one particular embodiment, may be interchanged or combined with alternate embodiments in various non-illustrated combinations or permutations.

It is also to be understood that, as used herein the terms “the,” “a,” or “an,” mean “at least one,” and should not be limited to “only one” unless explicitly indicated to the contrary. Thus, for example, reference to “an opening” includes examples having two or more such “openings” unless the context clearly indicates otherwise.

All scientific and technical terms used herein have meanings commonly used in the art unless otherwise specified. The definitions provided herein are to facilitate understanding of certain terms used frequently herein and are not meant to limit the scope of the present disclosure.

As used herein, “have,” “having,” “include,” “including,” “comprise,” “comprising.” or the like are used in their open-ended sense, and generally mean “including, but not limited to.”

Ranges may be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All numerical values expressed herein are to be interpreted as including “about” whether or not so stated, unless expressly indicated otherwise. It is further understood, however, that each numerical value recited is precisely contemplated as well, regardless of whether it is expressed as “about” that value. Thus, “a dimension less than 10 mm” and “a dimension less than about 10 mm” both include embodiments of “a dimension less than about 10 mm” as well as “a dimension less than 10 mm.”

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

While various features, elements or steps of particular embodiments may be disclosed using the transitional phrase “comprising.” it is to be understood that alternative embodiments, including those that may be described using the transitional phrases “consisting” or “consisting essentially of,” are implied. Thus, for example, implied alternative embodiments to a method comprising A+B+C include embodiments where a method consists of A+B+C, and embodiments where a method consists essentially of A+B+C.

Although multiple embodiments of the present disclosure have been described in the Detailed Description, it should be understood that the disclosure is not limited to the disclosed embodiments, but is capable of numerous rearrangements, modifications and substitutions without departing from the disclosure as set forth and defined by the following claims.

1. A protective carrier for a microcavity vessel comprising:
a substantially flat rigid plate comprising a top surface and a bottom surface;
a flange disposed around at least a portion of a perimeter of the rigid plate, the flange extending from the top surface of the rigid plate; and
one or more ribs disposed on the bottom surface of the rigid plate.

2. The protective carrier of claim 1, wherein the flange is disposed continuously around the perimeter of the top surface of the rigid plate.

3. The protective carrier of claim 1, wherein the flange is disposed around the perimeter of the top surface of the rigid plate on all but one side of the rigid plate.

4-5. (canceled)

6. The protective carrier of claim 1, wherein the one or more ribs disposed on the bottom surface of the rigid plate is proximate the perimeter of the rigid plate.

7. The protective carrier of claim 1, wherein the one or more ribs has a height of about 1 mm-2 mm extending from the bottom surface of the rigid plate.

8. The protective carrier of claim 1, further comprising a plurality of standoffs, wherein each standoff of the plurality of the standoffs is disposed at an interior surface of the flange on the top surface of the rigid plate.

9. (canceled)

10. The protective carrier of claim 8, wherein each standoff is an “L” shape, wherein a first portion is disposed vertically on an interior surface of the flange and a second portion is disposed horizontally on the top surface of the rigid plate.

11. The protective carrier of claim 10, wherein the first portion of each standoff has a height of about 3 mm-4 mm and a thickness of about 0.5 mm-1.5 mm.

12. The protective carrier of claim 10, wherein the second portion of each standoff has a length of about 3 mm-4 mm and a thickness of about 0.5 mm-1.5 mm.

13. The protective carrier of claim 8, wherein the protective carrier is configured to retain a bottom portion of a microcavity vessel, the bottom portion of the microcavity vessel disposed at the top surface of the rigid plate within the perimeter defined by the flange.

14. The protective carrier of claim 13, wherein the plurality of standoffs is configured to support the bottom portion of the microcavity vessel, allowing for air exchange to a microcavity substrate of the microcavity vessel.

15-16. (canceled)

17. The protective carrier of claim 1, wherein the protective carrier has a height of about 7 mm to 8 mm.

18. The protective carrier of claim 1, wherein a thickness of the rigid plate is about 1 mm-2 mm.

19. The protective carrier of claim 1, wherein the protective carrier is formed from a polymer, metal, or glass,
wherein the polymer comprises polystyrene, polypropylene, polyethylene polyethylene terephthalate, polymethylpentene, polycarbonate, polymethyl methacrylate, styrene-ethylene-butadiene-styrene, other such polymers, or a combination thereof,
wherein the metal comprises aluminum, stainless steel, zinc, or a combination thereof and
wherein the glass comprises a borosilicate glass.

20-22. (canceled)

23. The protective carrier of claim 1, wherein the protective carrier is formed from a recyclable or biodegradable material.

24-25. (canceled)

26. A protective microcavity vessel carrier system comprising:
a microcavity vessel comprising a microcavity substrate on a bottom surface; and
a protective carrier according to claim 1, and
wherein the microcavity substrate comprises a plurality of microcavities.

27. The system of claim 26, wherein the microcavity vessel is a microcavity flask, microcavity plate, microcavity bioreactor, or a stacked 3D microcavity culture vessel.

28-29. (canceled)

30. The system of claim 26, wherein each microcavity comprises a rounded bottom.

31. The system of claim 26, each microcavity is configured such that cells cultured in the microcavity vessel form three-dimensional (3D) cell aggregates.

32. The system of claim 26, wherein an interior surface of the microcavity substrate is non-adherent to cells.

33-35. (canceled)
